(19) United States
(12) Patent Application Publication
(10) Pub. No.: US 2013/0296834 A1
(43) Pub. Date: Nov. 7, 2013

(54) DEVICE FOR LASER-AIDED EYE SURGERY

(71) Applicants: WAVELIGHT GMBH, Erlangen (DE); ARMIN WELLHÖFER, Schwaig (DE); CHRISTOF DONITZKY, Eckental (DE); JOHANNES KRAUSE, Stein (DE)

(72) Inventors: Armin Wellhöfer, Schwaig (DE); Christof Donitzky, Eckental (DE); Johannes Krause, Stein (DE)

(73) Assignee: WaveLight GmbH, Erlangen (DE)

(21) Appl. No.: 13/872,697

(22) Filed: Apr. 29, 2013

(30) Foreign Application Priority Data
May 4, 2012 (DE) 10 2012 008998.8

(51) Int. Cl. A61F 9/008

(52) U.S. Cl. CPC A61F 9/00814; A61F 9/0084
USPC 606/5

(57) ABSTRACT

A device for laser-aided eye surgery comprising a patient table, as well as at least one laser system with a laser treatment head for emission of a focused laser beam onto an eye of a patient to be treated on the table. The laser system comprises an operation microscope with a viewing beam path running collinear to the laser beam path at least in the area of the emission location of the laser beam. According to the invention, the device is characterized by at least a first camera to create an image at least comprising the area from the mouth to the forehead of a patient lying on a table, as well as by a display device to display the picture taken. As an alternative to, or in addition to, the first camera and the display device, the eye-surgery device can comprise a laser projector for projection of a laser light pattern onto the patient's body as well as a guiding computer controlling the projector with an image signal output to which an image signal input of the projector is attached.

### TECHNICAL FIELD

The invention relates to a device for laser-aided eye surgery, comprising a patient table as well as at least one laser system with a laser treatment head for emission of a focused laser beam onto an eye to be treated of a patient on the table, wherein the laser system comprises an operation microscope with a viewing beam path running collinear to the laser beam path at least in the area of the emission location of the laser beam.

### BACKGROUND

For treatment of a human eye with laser radiation, generally the task is to position the eye precisely relative to the emission location at which the laser output emerges from the laser system. This holds true regardless of the type of treatment, be it an ablating treatment in which corneal tissue is removed by ablation, or whether it be an incision treatment, in which sections in the cornea or other parts of the eye are generated by photodisection, or whether it be some other laser-aided form of treatment.

Normally, laser systems used for eye treatments include an operation microscope through which the surgeon can observe the laser processing and can determine the precise positioning of the eye under the location of laser emission. However, if the eye must be aligned vis-à-vis the laser emission location, great difficulties are posed by solely using the operation microscope. Since the visual field which the surgeon has through the operation microscope is relatively small, the surgeon must repeatedly move away from the microscope to make sure that the patient is holding his head correctly, and to be sure that the correct eye is positioned under the laser treatment head in the first place.

For laser systems for incision processing, the patient's eye is coupled to the laser system with a special patient adapter, with the surgeon not, under certain circumstances, able to precisely see through the microscope in a certain range of approach of the patient adapter to the eye.

Through the microscope, the surgeon cannot with certainty determine if, for example, the nose or one of the bone areas (orbita) that border the eye socket of the human body are not lying in the approach path of the patient adapter. To avoid causing injuries to the patient, during the coupling process the surgeon must make a comparatively large number of head movements, to check the relative position between the patient's eye and the patient adapter, since as a rule it is covered by the laser device, for example a covering of the laser beam channel.

### SUMMARY

Therefore it is a task of the invention to provide the surgeon with suitable aids through which correct positioning of the eye to be treated beneath the laser emission location is facilitated for the surgeon.

### BRIEF DESCRIPTION OF THE DRAWINGS

The invention is explained in greater detail in what follows using the appended drawings. Shown are:

FIG. 1: a schematic view of a device for laser-aided eye surgery according to one embodiment example.

FIG. 2: a schematic view of a device for laser-aided eye surgery according to a second embodiment example.

FIG. 3: a schematic view of a device for laser-aided eye surgery according to a third embodiment example.

FIGS. 4 to 7: examples of laser light patterns that can be projected onto the patient's face with an eye-surgery device.

### DETAILED DESCRIPTION OF EXAMPLE EMBODIMENTS

To solve this problem according to the invention, a device of the type named at the outset is provided, which is characterized by at least a first camera to create an image at least comprising the area from the mouth to the forehead of a patient lying on a table, as well as by a display device to display the picture taken. The display device can, for example, comprise a screen for displaying the picture taken, with this screen being able to be situated so that the surgeon only needs to briefly look up from the microscope to observe the image shown on the screen. Alternatively or additionally, the display device can be designed for focusing the picture taken into the viewing beam path of the operation microscope. In this case the surgeon does not have to look up from the microscope to view the images of the first camera. These can, for example, be focused in an edge area of the viewing image which the surgeon sees through the microscope, or superimposed.

Preferably the first camera is directed toward taking images which detect the patient's entire head. On the images provided by the first camera, more parts of the patient's body can even be visible, such as the neck or chest area. In this way, the surgeon can readily detect whether, for example, the patient is leaning his head too far back or too close to his chest with his chin, or if the patient's head is inclined to the side. Alternatively or additionally, the first camera can be directed toward obtaining images that also detect at least a part of the laser treatment head, such as a patient adapter situated at the emission location of the laser output, and/or at least a part of a head support on the patient table. If in the images of the first camera, such a patient adapter is for example visible, the surgeon can detect the current relative position between the patient adapter and the eye to be treated, and thus determine if the coupling process is running properly or if the coupling has been properly concluded.

To detect sufficiently large areas around the patient's eye to be treated, it is recommended that the first camera exhibit a minimum viewing angle of greater than 25 degrees, for example more than 30 degrees or more than 35 degrees. For example, cameras obtainable commercially can be used which exhibit a minimum viewing angle of about 50 degrees or even 60 degrees, for example.

In a preferred embodiment, the invention-specific device in addition comprises at least one second camera separate from the first camera, to take images of the patient's eye to be treated, with the second camera being part of a beam-following mechanism, which detects the movements of the eye aided by sequences of images of the second camera, and which tracks the focal position of the laser output in dependence on the detected eye movements. Preferably the beam tracking mechanism operates independently of the images of the first camera, i.e., it detects movements of the patient's eye solely based on images of the second camera.

According to an advantageous configuration, the invention-specific device comprises a first laser system for carrying out tissue ablations on a human eye, as well as a second laser system for making tissue incisions in the human eye, with the wavelength of laser outputs emitted by the first laser system being below 300 nm, and the wavelength of laser outputs emitted by the second laser system above 300 nm, wherein the patient table is optionally able to be positioned under each of the two laser systems, and each laser system is equipped with at least a first camera. The first laser system with its output wavelength of less than 300 nm can be used for example for ablation of corneal tissue, such as in Lasik treatment, wherein, according to an ablation profile determined individually for the patient, corneal tissue is removed from the free front side of the stromal bed after pivoting away the previously generated Lasik flap. The second laser system with an output wavelength of above 300 nm can, in contrast, be used for incision tasks, such as for laser preparation of the above-mentioned Lasik flap, wherein the laser-induced optical penetration observable in dielectric materials is the decisive effect for generating the breakthrough.

Alternatively or in addition to a display of images from the first camera, an evaluation and control unit can be attached to the first camera, to subject the imagery of the first camera to an image evaluation. For example, in this regard an adjustment device connected with the evaluation and control unit can be provided for positional adjustment of the laser treatment head and/or of the patient table, depending on the results of the image assessment. It is also conceivable to provide a signal emission device controlled by the evaluation and control unit, to issue an optical and/or acoustic notification signal depending on the result of the image assessment. By means of such an image assessment, of the eye to be treated can be automatically positioned relative to the laser system, for example. Automatic detection can also be done of whether, for example, there are disturbing contours below the laser emission location, or whether the patient moves his head. In such a case, for example, an acoustic, haptic and/or optical warning message can be issued. With this, a warning tone, a staining or a corresponding notification in the operation microscope can be made, a warning light can go on, or a joystick provided for adjustment can vibrate.

As an alternative to, or in addition to, providing the first camera, the invention-specific device can comprise a laser projector for projecting a laser light pattern on the body, preferably at least onto the face of a patient lying on the table, as well as a guiding computer controlling the projector with a visual signal output to which a visual signal input of the projector is attached. At its visual signal output, the guiding computer can, for example, provide a standardized visual signal, such as according to the DVI standard, the VGA standard or the HDMI standard. By means of a projector thus guided, any laser light patterns per se can be projected onto the patient. Preferably the guiding computer is set up to issue visual signals at its visual signal output for one or more laser light patterns, which represent at least one of the following: an embodiment of a measure, a pattern for marking at least one anatomical structure of the patient's head, diagnosis information, patient identification information, an area of the eye to be processed by the laser beam, or a reference marking on the surface of the eye.

An example of an embodiment of a measure is a measurement scale projected on the patient, such as in the form of a dashed pattern or a dot pattern. Such an embodiment of a measure can serve for measuring of a patient (for example, the position of the head, the eyes, the viewing direction or the lid position), and then the laser system and/or the patient table and/or the patient himself can be suitably aligned to obtain an optimal position for laser treatment.

Diagnosis information and identifying information for the patient can be projected, for example, on the face or the torso of the patient. In a simple fashion, these permit the surgeon to obtain information about the patient lying on the table, to determine if the right operation is being conducted on the right patient. The identifying information can comprise, for example, the patient's name and/or a patient identifier code. The diagnosis information can for example comprise measured values and/or evaluation results, which were obtained during a prior diagnosis of the patient.

Patterns for marking anatomical structures on the patient's head can comprise, for example, crossed threads, a circular figure or some other conic section figure, lines, points or a polygonal figure. For example, such a marking pattern can form demarcation lines that are to be aligned vis-à-vis the outline contours of the patient's face. Alternatively or additionally, the marking pattern can mark the eye to be treated, by means of a rectangle or circle projected on the eye, for example, or around it, or crossed threads. If the projected laser light pattern pre-specifies in the manner explained a certain position, for example, of the patient's head using demarcation lines, and at the same time containing a special marking figure for the eye to be treated, first the surgeons, as per the eye measure can align the patient's head vis-à-vis the demarcation lines. Utilizing the special eye marking patterns, he then knows which eye is the one to be treated. If desired, he can make use of the projected eye marking pattern for fine-tuned alignment of the patient's head.

Alternatively or additionally, the projected laser light pattern can, with use of a laser, show an area of the eye to be processed, such as in the form of a circle corresponding to the size of an ablation area or an incision figure, or another geometric figure which is projected on the eye surface. Prior to the start of the operation, this allows the surgeon to do a (visual) check of whether the treatment plan is appropriate or whether possibly it needs to be revised. For example, it is conceivable for the surgeon to determine that an ablation area indicated through projection or a planned flap incision is too large due to the overall size of the patient's eye, and therefore needs to be reduced.

Alternatively, or in addition to, a projection figure representing the area of the eye to be treated, the laser light pattern can comprise a reference marking that is projected onto 6 the eye surface. For example, such a reference marking can depict a point which, with planned Lasik treatment, represents the intended location of the flap hinge. Also, such a projection of a reference marking on the eye surface can assist the surgeon in checking the intended treatment plan before the operation starts.

The projector can be installed on or in the laser system in fixed fashion, or alternatively it can be set up separate from the laser system. With installation in or on the laser system, the projector can possess a viewing beam path that runs collinear to the laser beam path at least in the area of the laser output emission. Alternatively, however, it is also conceivable to project the laser light pattern to be inclined from the side (related to the direction of the laser beam path from the emission location to the patient's eye), with the projector then possessing a projection beam path running totally separate from the laser output path.

In a preferred embodiment, the guiding computer is set up, in its image signal output, to optionally issue image signals for a plurality of different laser light patterns. For example, the guiding computer can execute a program that provides for automatic changeout between multiple laser light patterns which are projected onto the patient. The laser light patterns projected consecutively can be totally different; alternatively it is conceivable that the consecutively projected laser light patterns in part have identical components. The user can also undertake a changeout between different laser light patterns via a suitable user interface in instructable fashion.

The guiding computer can be set up to modify the projection of the laser light pattern depending on object movements which are detected by image assessment of pictures taken by the first camera. Detected object movements can, for example, be movements of the patient himself. The projection of the laser light pattern is tracked in correspondence to the motions of the patient. For the surgeon, this facilitates precise alignment of the patient and subsequent treatment. However, it is not precluded alternatively to track or generally to modify the projection of the laser light pattern, depending on detected movements, for example, of the surgeon himself or of the instrumentation used by him. To modify the projection of the laser light pattern, it is conceivable to arrange the projector to be mobile and to alter its position by means of a suitable drive mechanism. Likewise it is conceivable to arrange the projector in a fixed position, but to alter the image signal fed to it from the guiding computer in dependence on the detected object motions.

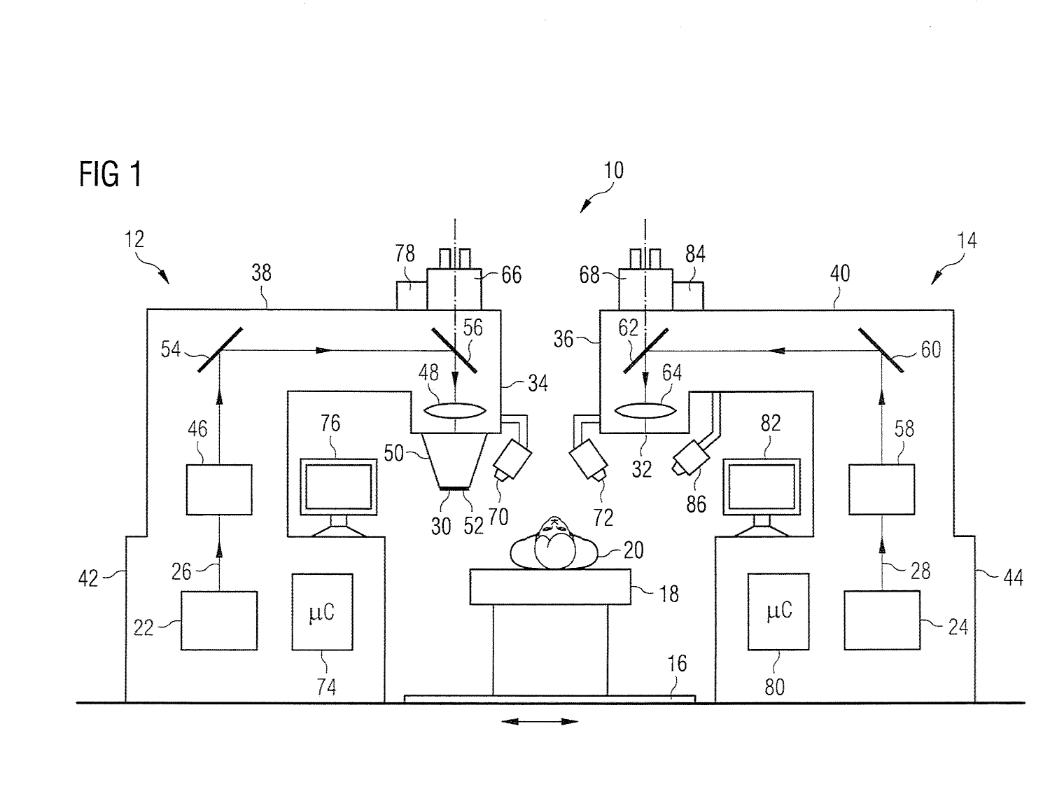

The device shown in FIG. 1 for laser-aided eye surgery—overall designated as 10—comprises two statically placed laser systems 12, 14 and a patient table 18 able to be moved on a ground rail 16 for a patient, whose head is schematically indicated at 20. The patient table 18 can be moved back and forth between the two laser systems 12, 14. The two laser systems 12, 14 each permit laser treatment of the patient's eye, but with different interactive processes being used and correspondingly different forms of treatment able to be carried out with the two laser systems 12, 14. Specifically, in the shown case example of FIG. 1, laser system 12 is designed to make tissue incisions in the human eye, with incisions primarily based on the effect of laser-induced optical breakthrough. For such a photodisruptive treatment of human eye tissue, as a rule lasing wavelengths of over 300 nm are required, wherein the lasing wavelength for example may be in the UV range or in the low IR range (for example, between 0.8 μm and 1.1 μm). The lasing pulse durations are for example in the nano-, pico-, femto- or atto-second range. In contrast, laser system 14 is designed for ablative eye treatment, in which the laser output with low transmission into the eye is largely absorbed on the surface of the irradiated tissue area and there results in a comparatively thin-layer tissue removal. The ablation lasers used regularly have a wavelength under 300 nm; for example, an ArF-excimer laser irradiating at 193 nm can be used.

Both laser systems 12, 14 comprise a particular radiation source 22 or 24, wherein the laser output emitted by the source—designated by 26 and 28 —is guided by devices for beam guidance and formation to a beam emission location 30 and 32, where the laser beam in question emerges downward, essentially vertically. The emission locations 30, 32 are on a laser treatment head 34 or 36 of the laser system in question, wherein the laser treatment heads 34, 36 are situated at the end of a ray arm 38 or 40 that is for example jointed and preferably movably arranged. The ray arms 38, 40 for their part extend from a device main body 42 and 44 of the laser system in question.

In laser system 12, the devices mentioned for beam guidance and shaping comprise, among other items, a scanner device 46 for three-dimensional adjustment of the focal position of laser output 26 as well as a focusing lens 48, often consisting of multiple lenses, situated in laser treatment head 34, which is schematically shown in FIG. 1 as a single lens for illustration reasons. Next to the focusing lens 48, on the laser treatment head 34, is a patient adapter 50 with a contact element 52 that is transparent or translucent to the laser output, the underside of which is meant to be in contact with the eye to be treated. Deflecting mirrors 54, 56 guide the laser output 26 in the ray arm 38.

Laser system 14 comprises a scanner device 58, by means of which the focal position of laser output 28 can be adjusted in a direction transverse to the lasing propagation direction (a so-called x-y scanner). In contrast, longitudinal scanning of the laser position in the lasing propagation direction is often not required with an ablation laser in view of the typically comparatively small numerical aperture of the focused beam cluster directed to the eye and the corresponding comparatively large Rayleigh length. It is not precluded to also equip scanner device 58 with a longitudinal scanning capability. Deflection mirrors 60, 62 direct the laser output 28 in turn to an often multi-lens focusing lens 64, which is however schematically depicted in FIG. 1 only as a single lens, from which the laser output 28 is emitted essentially vertically downward from laser treatment head 36.

In a variation from the depiction in FIG. 1, the scanner devices 46, 58 can be placed in the beam path of the particular laser output after the two deflecting mirrors 54, 56 or 60, 62, thus directly in front of focusing lens 48 or 64.

On floor rail 16, the patient table 18 can be moved under each of the laser treatment heads 34, 36, so that the patient with his head 20 is vertically beneath the laser treatment head in question. In the case of laser system 12, for conducting a laser treatment, the patient with the eye in question is to be connected to patient adapter 50. Here it is conceivable that the ray arm 38 and/or the patient table 18 are adjustable in height. It is desirable that ray arm 38 also be horizontally adjustable, to make possible a precise alignment of patient adapter 50 vis-à-vis the patient's head 20. Also with laser system 14 it is conceivable that ray arm 40 be able to be adjusted in a vertical and horizontal direction.

In addition, laser system 12 is equipped with an operation microscope 66, through which the surgeon can look at the beam emission location 30. It is perceptible that for this the viewing beam path of microscope 66 runs collinear to the beam path of laser output 26, in any case in the area from deflection mirror 56 to beam emission location 30. If the patient is connected to patient adapter 50, the surgeon can also observe the eye in question through microscope 66.

Another operation microscope 68 is provided on laser system 14. Microscope 68 has a viewing beam path that runs collinear or at an angle to the beam path of laser output 28 (in the area from deflection mirror 62 to beam emission location 32), permitting the surgeon to have an enlarged view of the beam emission location 32 and the area under it.

While conducting a laser treatment with one of laser systems 12 or 14, the surgeon sits (or stands) behind the patient's head, for example, and looks through the microscope 66 or 68 in question. To facilitate correct alignment of the patient relative to the particular laser system 12 or 14, both laser systems 12, 14 are equipped with a camera 70 or 72, which can take images of the patient's head 20 and also if necessary of parts of the patient table 18 and/or —in the case of laser system 12—of parts of patient adapter 50. Cameras 70, 72 are attached on the laser system in question, for example in the area of the particular laser treatment head 34 or 36, so that they have the desired areas of the patient, such as the head 20, in the visual field, when the patient with his head is roughly aligned vertically under the laser treatment head 34 or 36 in question. The viewing angle of cameras 70, 72 can be large enough that if the patient 11 table 18 and/or the laser treatment head 34 or 36 are adjusted in height, the patient's head 20 remains in the visual field of camera 70 or 72. Cameras 70, 72 both constitute a first camera in the meaning of the invention.

The images of camera 70 are transferred by a central evaluation and control unit 74 of laser system 12 to a screen 76 for display and/or delivered to a beam collimator (head-up display) 78 coupled with microscope 66, which causes a collimation of the image taken by camera 70 into the viewing beam path of microscope 66. Evaluation and control unit 74 governs all controllable functions of laser system 12, such as also the operation of radiation source 22 and scanner device 46.

In a similar manner, laser system 14 is designed with a central evaluation and control unit, which is designated by 80 in FIG. 1. This can bring the images taken by camera 72 to a screen 82 for display, or provide them to a beam collimator 84 coupled with microscope 68, so that the surgeon can observe an occurrence recorded by camera 72, without having to take his eyes away from microscope 68. Evaluation and control unit 80 governs all the controllable functions of laser system 14, such as operation of the radiation source 24 and of scanner device 58.

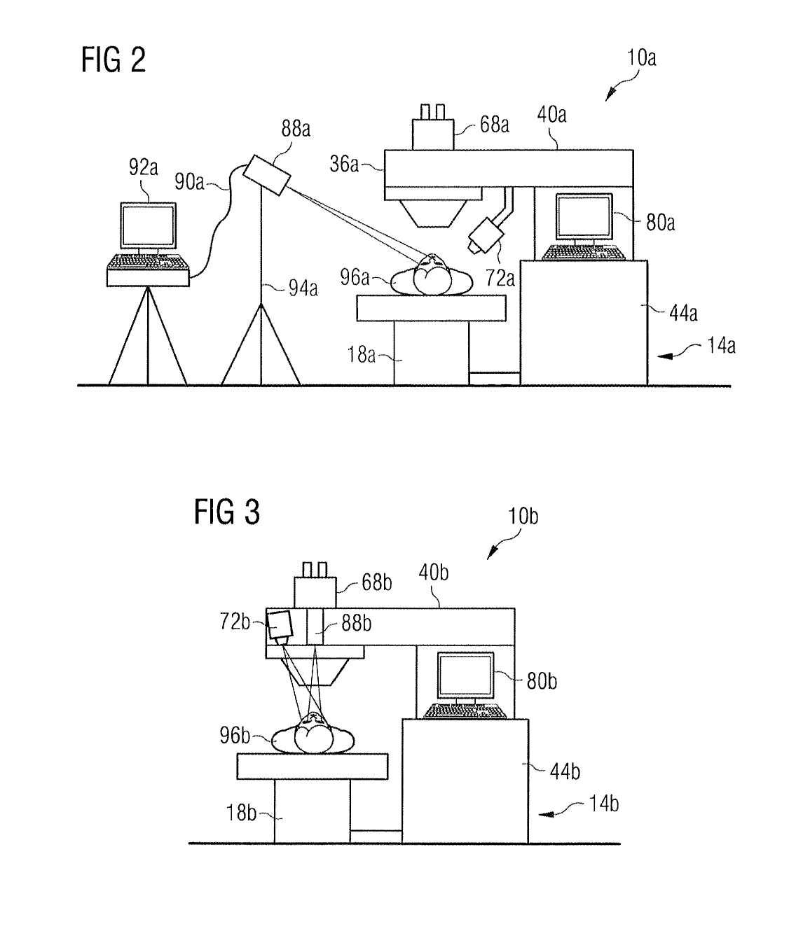

Screens 76, 82 are positioned so that they are in the immediate field of the surgeon's vision, as soon as the surgeon takes his eyes away from microscope 66 or 68, so that for this the surgeon needs to move his head only relatively slightly. In a variation of the depiction in FIG. 2, the screens 76, 82 can be situated above on the ray arm 38 or 40, thus roughly at the height of the particular operation microscope 66 or 68.

Because there is no direct mechanical coupling between the patient's eye and laser system 14 when using laser system 14, in the example case of FIG. 1, the laser system 14 comprises an additional camera 86, which, in comparison to cameras 70, 72, possesses a considerably smaller viewing angle of only a few degrees, for example, and which takes images of the eye to be treated. The images of camera 86 are evaluated by unit 80 to detect movements of the eye. By suitable guidance of scanner device 58, unit 80 can track laser output 28, i.e. the beam focus, in dependence on the detected eye movements. Thus, together with the pertinent image evaluation software in evaluation and control unit 80, camera 86 constitutes an eye tracker, which, for example, tracks the position of the pupil center. The images of camera 72 are not used for this eye tracking. Camera 86 constitutes a second camera in the sense of the invention.

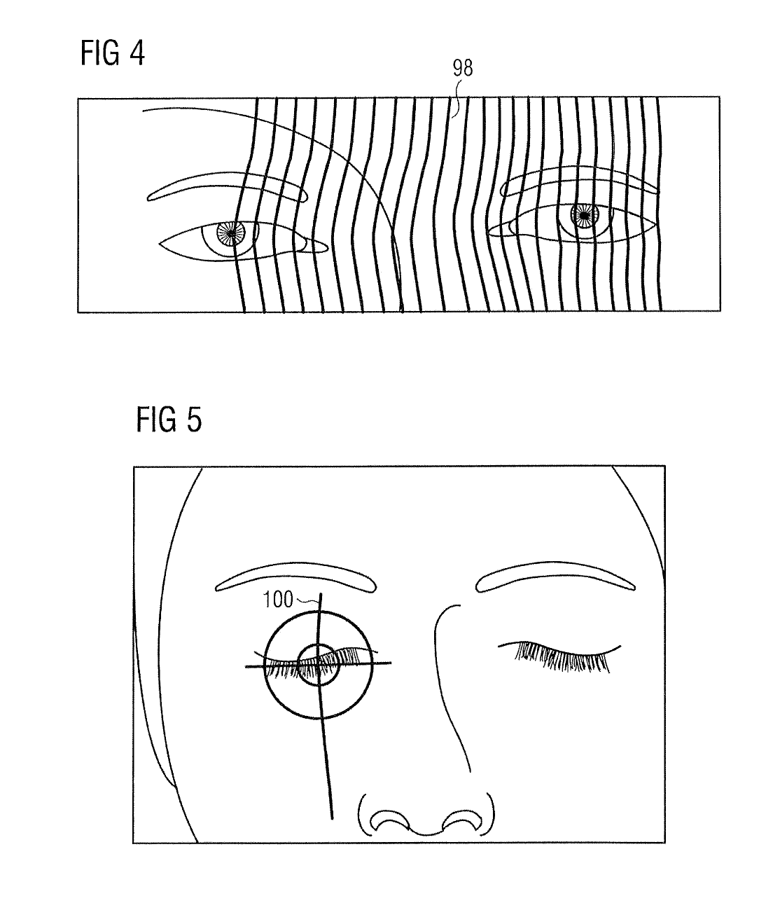

In FIGS. 2 and 3, components that are the same as in FIG. 1, or act the same, are designated with the same reference numbers, but supplemented by a lower-case letter. Since only a single laser system is shown in FIGS. 2 and 3, the corresponding reference symbols of laser system 14 of FIG. 1 are drawn upon, but this is not to be understood as a limitation to a certain form of treatment such as ablation.

In the embodiment example as per FIG. 2, the eye-surgery device 10a comprises, in addition to laser system 14a, a laser projector 88a, which is attached via a cable 90a to a projector guiding computer 92a. Projector 88a stands on a stand (here a tripod) 94a, which can be set up independent of laser system 14a at a freely-selected location in the treatment room. From projector guiding computer 92a, it receives via cable 90a an image signal, which represents a laser light pattern to be projected. By means of base 94a, projector 88a can be so aligned that the laser light pattern can be projected on the patient—designated by 96a—and, for example, onto his face and/or trunk. The surgeon can see the projected laser light pattern by turning his gaze from microscope 68a and looking laterally past laser treatment head 36a at the patient. Alternatively or additionally, laser system 14a can comprise a camera 72a, to make an image of the patient's head, and, if desired, additional patient body parts, and this image, in which the laser light pattern is perceptible, is collimated for example in the viewing beam path of microscope 68a.

In the version as per FIG. 3, laser projector 88b is incorporated in fixed fashion into laser system 14b, with the laser light pattern being projected, for example, in collinear fashion to the beam path of the processing laser output onto the patient. Alternatively, also with a fixed installation of laser projector 88b, projection of the pattern at a slant from the side onto patient 96b is conceivable.

A separate projector guiding computer for guiding laser projector 88b can be dispensed with in the embodiment form as per FIG. 3. In this case, the central evaluation and control unit 80b can in this case control projector 88b and its power supply with a suitable image signal.

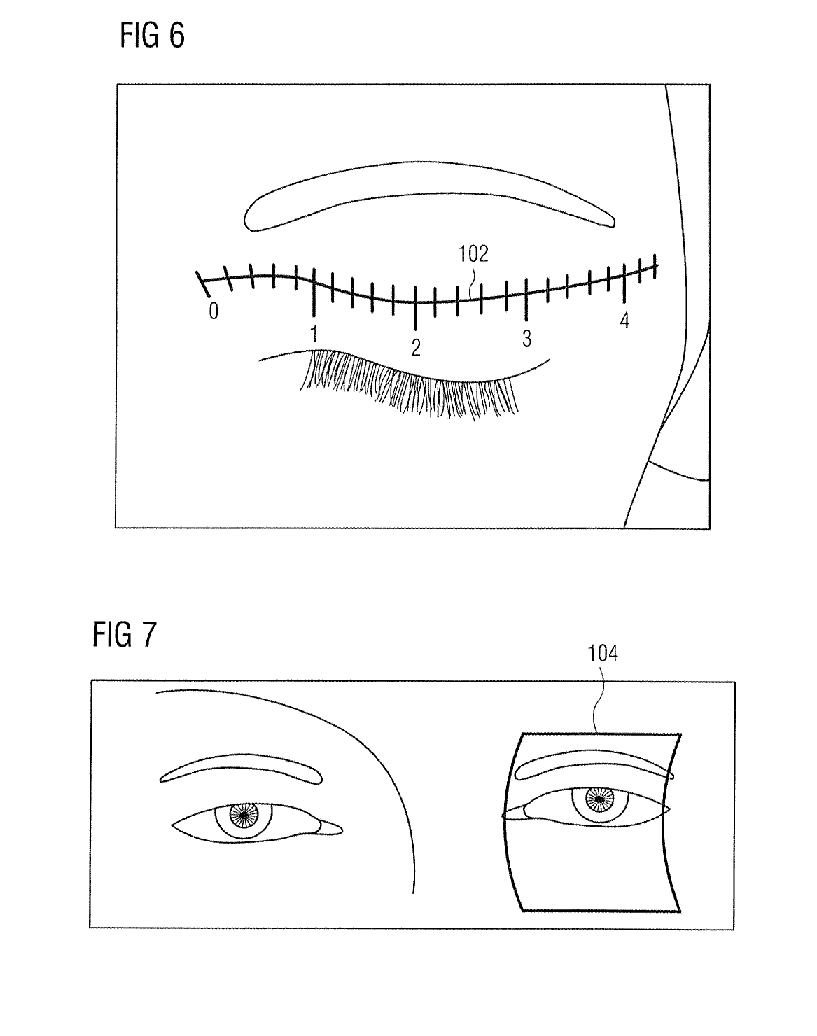

Various versions of laser light patterns, which can be projected onto the patient's face, are shown in FIGS. 4 to 7.

FIG. 4 shows a stripe pattern 98 consisting of a plurality of straight line stripes running parallel next to each other, which can be projected on the face and there for example onto the eye part.

FIG. 5 depicts the projected laser light pattern of a crosshairs FIG. 100, which in the exemplary example depicted comprises two straight light strips crossing each other at right angles as well as two circular light strips concentric to the crossing point of the two straight lines.

FIG. 6 shows an example of the embodiment of a measure projected onto the face of a patient in the form of a scale 102, which can for example represent a cm-scale.

Lastly, FIG. 7 shows an example with a rectangular light pattern 104, which are projected onto the face of the patient so that it marks the eye to be treated. It is understood that other geometric forms can be used to emphasize the particular eye that is to be treated.

1. Device for laser-aided eye surgery, comprising a patient table as well as at least one laser system with a laser treatment head for emission of a focused laser output onto an eye to be treated of a patient on the table, wherein the laser system comprises an operation microscope with a viewing beam path running collinear to the laser beam path at least in the area of the emission location of the laser output, characterized by at least a first camera to create an image at least comprising the area from the mouth to the forehead of a patient lying on a table, as well as by a display device to display the picture taken.

2. Device according to claim 1, wherein the display device comprises a screen for displaying the picture taken.

3. Device according to claim 1, wherein the display device is configured for collimation of the picture taken in the viewing beam path of the operation microscope.

4. Device according to claim 1, wherein the first camera exhibits a minimum viewing angle of more than 25 degrees, for example more than 30 degrees or more than 35 degrees.

5. Device according to claim 1, wherein the first camera set up to acquire images which detect the patient's entire head.

6. Device according to claim 1, wherein the first camera is set up to acquire images, which also detect at least a part of the laser treatment head, especially a patient adapter placed at an emission location of the laser output, and/or at least a part of a head rest of the patient table.

7. Device according to claim 1, comprising at least one second camera separate from the first camera for acquiring an image of the patient's eye to be treated, wherein the second camera is part of a beam tracking mechanism, which using a sequence of images of the second camera, detects movements of the eye and tracks the focal position of the laser output in dependence on the detected eye movements, wherein the beam tracking mechanism operates independent of the images of the first camera.

8. Device according to claim 1, comprising a first laser system for conducting tissue ablations on the human eye as well as a second laser system for generating tissue incisions in the human eye, wherein the wavelength of the laser output emitted by the first laser system is below 300 nm and the wavelength of the laser output emitted by the second laser system is above 300 nm, wherein the patient table is optionally able to be positioned beneath the laser treatment head of both laser systems, and each laser system is equipped with at least a first camera.

9. Device according to claim 1, comprising an evaluation and control unit attached to the first camera to conduct an image assessment on the images of the first camera.

10. Device according to claim 9, comprising an adjustment device connected with the evaluation and control unit for positional adjustment of the laser treatment head and/or the patient table in dependence on the result of image analysis.

11. Device according to claim 9, comprising a signal emission device guided by the evaluation and control unit for issuance of an optical and/or acoustic notification signal in dependence on the result of image analysis.

12. Device according to claim 1, comprising a capacitive sensor technology for sensory detection of the patient lying on the patient table, wherein the sensor technology is connected with an evaluation and control unit, which is set up to emit an optical and/or acoustical and/or haptic notification signal in dependence on a sensor signal of the sensor technology.

13. Device according to claim 1, further comprising a laser projector for projecting a laser light pattern on the body, preferably at least on the face of the patient lying on the table, as well as a guiding computer controlling the projector with a visual signal output to which a visual signal input of the projector is attached.

14. Device according to claim 13, wherein the projector is installed on or in the laser system or can be set up separate from the laser system.

15. Device according to claim, wherein the projector possesses a projection beam path running totally separate from the laser output path.

16. Device according to claim 13, wherein the projector possesses a projection beam path that at least in the area of the laser output emission location runs collinear to the laser output path.

17. Device according to claim 13, wherein the guiding computer is set up optionally at its image signal output to emit visual signals for a plurality of different laser light patterns.

18. Device according to claim 13, wherein the guiding computer is set up at its image signal output to issue visual signals for one or more laser light patterns which represent at least one of the following: an embodiment of a measure, a pattern for marking at least one anatomical structure of the patient's head, diagnosis information, patient identification information, an area of the eye to be processed by the laser beam, a reference marking on the surface of the eye.

19. Device according to claim 13, wherein the guiding computer is set up to modify the projection of the laser light pattern in dependence on object movements, which are detected by image assessment of the images taken by the first camera.
